(19) United States
(12) Patent Application Publication
(10) Pub. No.: US 2006/0069043 A1
(43) Pub. Date: Mar. 30, 2006

(54) Chiral separation, characterization and biological action of optically active isomers of digoxin

(76) Inventors: John Charin Somberg, Lake Forest, IL (US); Vasant V. Ranade, Libertyville, IL (US)

Correspondence Address: GILDO E. FATO, ESQUIRE;c/o LAURENCE M. DUNLAP, LTD., SUITE 202B, 14047 PETRONELLA DRIVE, LIBERTYVILLE, IL, 60048, US

(21) Appl. No.: 10/951,900

(22) Filed: Sep. 29, 2004

(51) Int. Cl. C07J 17/00; A61K 31/704

(52) U.S. Cl. 514/26; 536/6.1

(57) ABSTRACT

Disclosed herein is the method for separation of enantiomers or isomers of digoxin. These isomers are to be used in the treatment of heart failure without adverse or unneeded cardiac actions in humans. Additionally what is claimed is an isolate with less or no cardiac contractile effect but with AV node slowing such that the composition would be an effective therapy for the control of the ventricular response in atrial fibrillation. Also disclosed are methods for assaying these isomeric compounds present in biological fluids to enable the separation of pharmacologic actions.

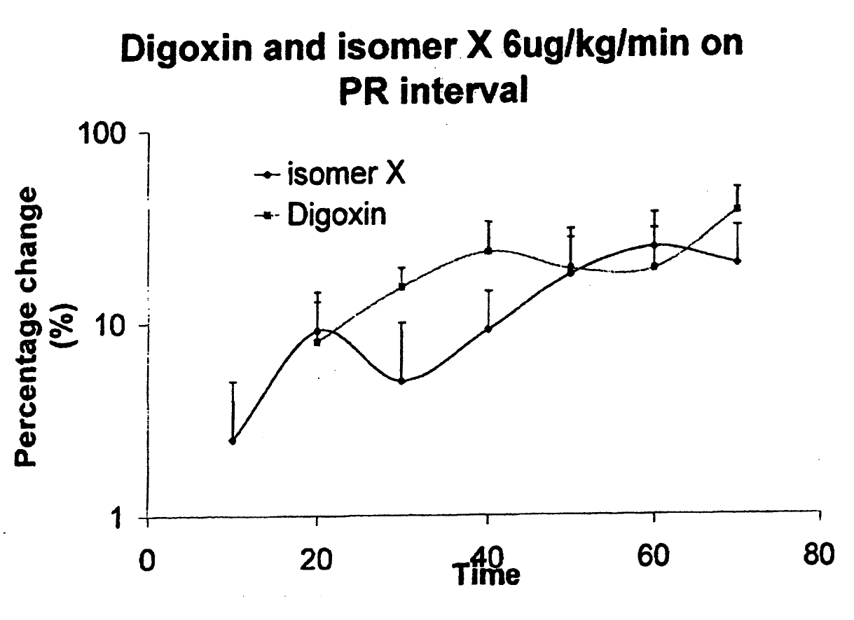

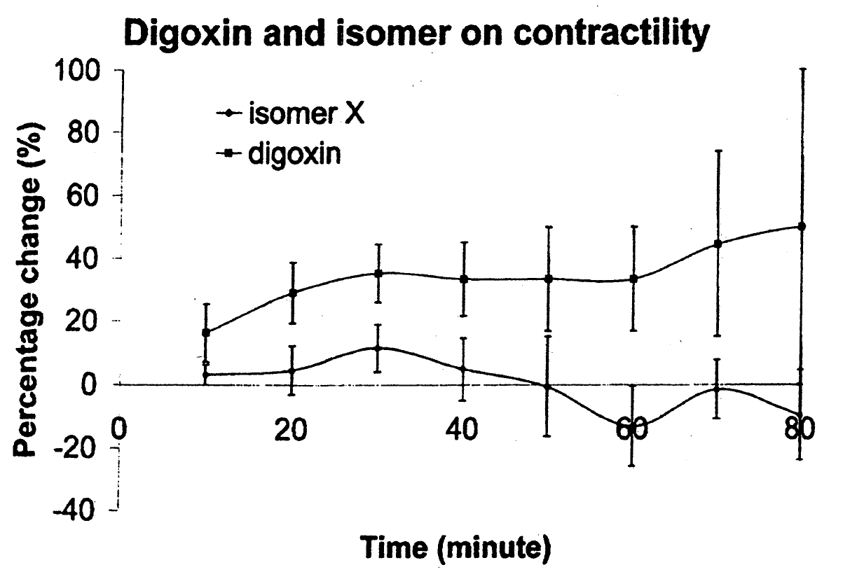

### BACKGROUND OF THE INVENTION

### FIELD OF THE INVENTION

The present invention relates to cardiotonic compounds, compositions and methods, more specifically of compounds that are optically active isomers of digoxin.

### INTRODUCTION

Digoxin, a digitalis glycoside, is widely used for the treatment of atrial fibrillation and heart failure. Digoxin has been used for years to prolong conduction of the AV node. Digoxin has positive inotropic and negative chronotropic actions, as well as a number of unwanted side effects, such as the facilitation of cardiac arrhythmias through its action on the autonomic nervous system and calcium transients in the myocardium. The primary molecular action of digoxin is the inhibition of sodium potassium ATPase and, through this inhibition, it is believed the AV node and inotropic effects are achieved. Recent studies have found that there are a number of isoforms of the receptor of digoxin action, NaK-ATPase in myocardium and in the AV node. Digoxin has several chiral centers; therefore, theoretically, a molecule of digoxin exists as a mixture of several enantiomers. These stereoisomers may possess differential effects on the SA and AV nodes, as well as on cardiac myocites to a varying degree.

Recently, there has been considerable interest in preparing and testing enantiomers of drugs that exert their pharmaceutical action via specific receptors or enzymes. In many instances, this has been shown to result in enhanced activity, greater potency and fewer side effects. Specifically, the anti-histamine terfenadine caused QT prolongation, while its chiral metabolite did not. While the prokinetic agent cisapride causes QT prolongation and arrhythmias, the chiral isomer nor-cisapride does not. In the case of floxacins, e.g. ofloxacin, optically active enantiomers have been separated and isolated by HPLC. (−)-Ofloxacin is about 8-128 times more active as the racemate against both gram-negative and gram-positive bacteria. Subsequent biotesting has demonstrated that the S-antipode was the more active both in bacteria and in cell-free enzyme assays. Chiral isomers of the floxacin may exhibit similar or more potent antibiotic activity, has no effect or a decreased effect on the QT interval, and produce less or no cardiac arrhythmias.

Previously, differences in the pharmacological activity and pharmacokinetic behavior between enantiomers were demonstrated in the case of beta-blockers, e.g. levalbuterol and beta-amino alcohols; amphetamine (AP); methamphetamine (MAP); and pencillamine. R- and S-isomer of AP and MAP have been known for sometime with the S-isomer being approximately five times more active than the R-isomer in their effects on the CNS. Recently, similar differences have become evident in the case of antihistamine terfenadine and antihistaminic antidepressant, fexofenadine. In the case of fluoxetine (Prozac®), a single-isomer preparation is under development and, in the case of cetirizine (Xyrtec®), a single isomer version is now available. A single-isomer version of cisapride (Norcisapride®) has a different receptor binding profile than the parent drug. Preliminary data on the pharmacodynamics of enantiomers have indicated that one single-isomer version can significantly reduce, if not eliminate, drug interaction; possess differences in receptor binding profiles; and exhibit different absorption, distribution, and metabolism properties.

The results with terfenadine indicated that the R-enantiomer of an orally administered racemate was preferentially oxidized in rats to form a carboxylic acid metabolite enriched in the R-enantiomer. The enantiomer is now marketed by Hoechst Marion Roussel as Allegra® that has a binding profile different from that of the parent drug. Preliminary data regarding the pharmacodynamics of enantiomers have indicated that one single isomer version can significantly reduce, if not eliminate, drug interactions; possess differences in receptor-binding profiles; and follow a different course in absorption, distribution, metabolism, excretion, and toxicokinetics.

In the class of antihyperlipidemic agents, atorvastatin was shown competitively to inhibit 3-hydroxy-3-methylglutaryl-coenzyme A (HMG-CoA) reductase, the enzyme that catalyzes the conversion of EMG-Co A to mevalonate. This has been the early rate-limited step in the cholesterol biosynthesis. This statin increases HDL-cholesterol, and decreases LDL-cholesterol, VLDL-cholesterol and plasma tryglycerides. Lipitor®, the R(+) isomer of atorvastin is a very effective, potent and a top-selling antihyperlipidemic agent. S(−) isomer of atorvastin was obtained by stereospecific synthesis. However, this isomer was found to be less potent than Lipitor®, the R(+) isomer.

### DESCRIPTION OF THE RELATED ART

Digoxin is a glycoside possessing desirable cardiac activity. However, routine therapeutic use of this drug is limited because of its adverse cardiac effects, such as HR slowing, heart block, and coronary and systemic vasoconstriction, and increased O₂ consumption due to increased cardiac work. Cardiac arrhythmias increase in patients who receive digoxin, in patients who have a myocardial infarction, have heightened adrenergic stimulation, or are hypoxic. Since digoxin has several optically active centers, separation of these enantiomers and testing of these compounds may afford the development of digoxin isomers with either reduced undesirable adverse effects or with complete absence of these adverse actions. The primary aim is to isolate one isomer or mixture of isomers that has a predominate effect on AV node slowing and one isomer or group of isomers with a predominantly inotropic action. The present invention also provides a method to assay and to provide possible quantitative determination of quantities of optically active isomers of digoxin in biological fluids and correlate this with their differential effects on the AV node and cardiac contractibility.

Qazzaz et al., 1999, reported on the two biologically active isomers of dihydroouabain. Ouabain is also a cardiac glycoside, and it is structurally related to digoxin. In the case of dihydroouabain, catalytic hydrogenation of the lactone ring linkage yielded two isomeric compounds. Each of these compounds had a different potency for inhibition of sodium pump activity. Similarly, in the case of digoxin, hydrogenation of the double bond present in the lactone can result in the introduction of an extra asymmetrical chiral center at C-20, thus leading to the formation of two isomeric compounds. In 1985, however, Reuning et al reported that these isomeric compounds were formed by gastrointestinal bacteria in patients on digoxin therapy, and the predominant epimer found in humans after this therapy was the 20R-isomer. Our results on the chiral separation of digoxin isomers could possibly indicate their potential use as model compounds in characterizing endogenous cardenolide-like factors found in mammals. These compounds have potential in studies characterizing kinetics of the inhibition of the sodium pump and studying membrane current potential as well.

### SUMMARY OF THE INVENTION

The present invention is related to isomers of digoxin, and the compositions and methods for testing these enantiomers for cardiotonic activity. The compounds are optically active digoxin isomers. Accordingly, in a broad aspect, the invention provides compounds of Formula I that contain stereoisomers.

Probable Structures of Digoxin Enantiomers:

Formula I 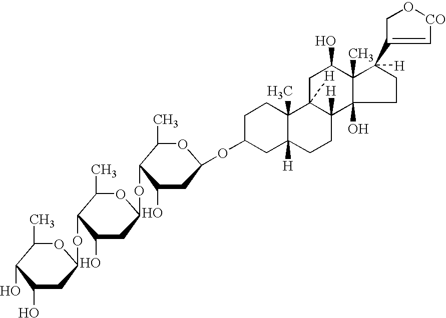
Description

Digoxin is a cardiotonic glycoside obtained from the leaves of Digitalis lanata (Fam. Scrophulariaceae).

Names

3β-[(0-2,6Dideoxy-β-D-ribo-hexopyranosyl-1 (1→4)-0-2,6-dideoxy-β-D-ribo-hexopyranosyl)oxyl-12β,14-dihydroxy-5β-card-20(22)-enolide

Cordioxil, Davoxin, Digoacin, Dilanacin, Disina, Lenocardin, Lanicor, Lanoxin, Rougoxin, Vanoxin (for example)

Formula, Structure, Molecular Weight

C₁₄H₆₄O₁₄ (Mol wt 780.96)

### DETAILED DESCRIPTION OF THE INVENTION

The present invention provides compounds, compositions and methods of use for optically active digoxin isomers or mixtures of isomers in the treatment of congestive heart failure, atrial fibrillation and/or atrial flutter or other superventricular arrhythmias.

After separation, purification and characterization, each individually identified isomer will be tested initially for the following criteria: differential effects on AV node conduction and cardiac compatibility.

Additional mixtures of isomers will be separated, optically identified, characterized by their differential effects on cardiac contractibility, and on blocking or slowing conduction at the AV node in the heart.

The compounds of the invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. In addition, there is provided a pharmaceutical formulation comprising a compound of the present invention and a pharmaceutically acceptable carrier. One or more compounds of the invention may be present in association with one or more non-toxic pharmaceutically acceptable carriers and/or diluents and/or adjuvants and, if desired, other active ingredients. The pharmaceutical compositions containing compounds of the present invention may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically acceptable and palatable preparations. Tablets contain the active ingredient in admixture with non-toxic pharmaceutically acceptable excipients which are suitable for the manufacture of tablets. These excipients may be, for example, corn starch or alginic acid; binding agents, for example, starch, gelatin or acacia; and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin; or as soft gelatin capsules wherein the active ingredient is mixed with water or an oil medium, for example, peanut oil, liquid paraffin or olive oil.

Aqueous suspensions contain the active materials in admixture with excipients suitable for the manufacture of aqueous suspensions. Such excipients are suspending agents, for example, sodium carboxymethylcellulose, methylcellulose, hydropropylcellulose, sodium alginate, polyvinylpyrrolidone, gum tragacanth and gum acacia; dispersing or wetting agents may be a naturally-occurring phosphatide, for example, lecithin; or condensation products of an alkylene oxide with fatty acids, for example, polyoxyethylene stearate; or condensation products of ethylene oxide with long chain aliphatic alcohols, for example, heptadecaethyleneoxycetanol; or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example, polyethylene sorbitan monooleate; the aqueous suspensions may also contain one or more preservatives, for example, ethyl or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example, arachis oil, olive oil, sesame oil, coconut oil, or in a mineral oil such as liquid paraffin. The oily suspensions may contain a thickening agent, for example, beeswax, hard paraffin or cetyl alcohol. Sweetening agents, such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present. Exceptions may be added to accelerate dissolution when taken sublingually or orally to provide rapid absorption.

Pharmaceutical compositions of the invention may also be in the form of oil-in-water emulsions. The oily phase may be a vegetable oil, for example, olive oil or arachis oil; a mineral oil, for example, liquid paraffin; or mixtures of these. Suitable emulsifying agents may be naturally-occurring gums, for example, gum acacia or gum tragacanth; naturally-occurring phosphatides, for example, soybean, lecithin, and esters or partial esters derived from fatty acids; hexitol, anhydrides, for example, sorbitan monooleate; and condensation products of the said partial esters with ethylene oxide, for example, polyoxyethylene sortitan monooleate. The emulsions may also contain sweetening and flavoring agents.

Syrups and elixirs may be formulated with sweetening agents, for example, glycerol, propylene glycol, sorbitol or sucrose. Such formulations may also contain a demulcent, a preservative and flavoring and coloring agents. The pharmaceutical compositions may be in the form of a sterile, injectable aqueous oleaginous suspension. This suspension palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present. Exceptions may be added to accelerate dissolution when taken sublingually or orally to provide rapid absorption.

Pharmaceutical compositions of the invention may also be in the form of oil-in-water emulsions. The oily phase may be a vegetable oil, for example, olive oil or arachis oil; a mineral oil, for example, liquid paraffin; or mixtures of these. Suitable emulsifying agents may be naturally-occurring gums, for example, gum acacia or gum tragacanth; naturally-occurring phosphatides, for example, soybean, lecithin, and esters or partial esters derived from fatty acids; hexitol, anhydrides, for example, sorbitan monooleate; and condensation products of the said partial esters with ethylene oxide, for example, polyoxyethylene sortitan monooleate. The emulsions may also contain sweetening and flavoring agents.

Syrups and elixirs may be formulated with sweetening agents, for example, glycerol, propylene glycol, sorbitol or sucrose. Such formulations may also contain a demulcent, a preservative and flavoring and coloring agents. The pharmaceutical compositions may be in the form of a sterile, injectable aqueous oleaginous suspension. This suspension may be formulated according to the known art using those suitable dispersing or wetting agents and suspending agents which have been mentioned above. The sterile, injectable preparation may also be sterile injectable solution or suspension in a non-toxic parentally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, fatty acids, such as oleic acid, find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compounds of the present invention may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of-the above-indicated conditions (about 0.5 mg to about 7 g per patient per day). The amount of active ingredient that may be combined with the carrier materials to produce a single dosage form will vary depending upon the species of the host animal to be treated, the particular mode of administration, and the body weight of the host. Dosage unit forms will generally contain from about 1 mg to about 500 mg of an active ingredient.

It will be understood, however, that the specific dose level for any particular patient will depend upon a variety of factors, including the activity of the specific compound employed, the age, body weight, general health, sex and diet of the patient, time of administration, route of administration, and rate of excretion, drug combination and the severity of the particular disease undergoing therapy.

For administration to non-human animals, the composition may be added to the animal feed or drinking water. It will be convenient to formulate these animal feed and drinking water compositions with a pellet-dose of the drug so that the animal takes in an appropriate quantity of the composition along with its diet. It will also be convenient to present the composition as a premix for addition to the feed or drinking water.

The following examples are illustrative of the invention but do not serve to limit its scope.

Experimental Section

### EXAMPLE 1

### Isomer Separation Methodology

The aim is to collect fractions of the eluent containing isomers after chromatographic separations. The chiral columns have been found to effectively separate stereoisomers and have proven to be effective tools in determining enantiomeric purity. Generally, resolution can be optimized by altering the mobile phase composition and/or by selecting chiral columns with specific packing materials. Separations are performed using non-polar organic phases (e.g., heptane, iso-octane) with polar organic additives, such as tetrahydrofuran, alcohols, chlorinated hydrocarbons or similar solvents with or without buffer such as phosphate or borate. Often, addition of a small amount of strong acids (e.g., TFA) to the mobile phase will considerably improve separation of the isomers. Anion exchange chromatography with aqueous buffers using salt or pH gradients can also be effectively used with silica-based packing in the columns that are covalently bonded with a polymeric polyethyleneimine network that is stable in both acidic and weakly basic eluents.

### EXAMPLE 2

### Chiral Chromatography of Digoxin

Chromatographic separations were achieved using two cyclobond I 2000 columns attached one after the other. The specifications of the column are particle size 5 μm spherical and 250×4.6 mm dimensions (Alltech Associates, Deerfield, Ill.). The solvent system used was CH₃CN: (1:3, V/V, pH 7.1) and the flow rate was 1 ml/min. The UV detector was set at 220 nm and the Spectra Physics HPLC (Solvent System P2000) instrument with Spectra 2000 programmable wave length detector and chromjet integrator was used. The digoxin solution was made as 1 mg/ml in ethanol and a sample of 10 μl was injected onto the column and the chromatograms were recorded. The Chromatogram indicated two peaks at 5 and 6 minutes. The recovery of the sample was quantitative (Ave 99%), indicating that these are only two isolates in this chiral separation. Mass spectra of the isolates indicated HPLC had been performed on the digoxin samples, indicating presence of digoxin; any other related compounds were not present. Using columns such as Lichrosorb S160, Merckosorb 5160, Ultrasphere Si, Nucleosil C₁₈, Whatman ODS-1, Perisorb RP and Zorbax Sil with a variety of solvent systems as the mobile phase, digoxin and the isolates can be detected.

Waters Alliance 2690 HPLC Pump Initial Conditions for Chiral Separation of Digoxin:

| Solvents | | |
|---|---|---|
| A % | 0.0 | |
| B % | 100.0 | Digoxin Mobile Phase |
| C % | 0.0 | Methanol |
| D % | 0.0 | Solvent D |
| Flow Ramp | 0.50 | |
| Flow (ml/min) | 0.400 | |
| Stop Time (mins) | 20.0 | |
| Column Temperature (° C.) | 20.0 | |
| Column Temperature Limit (° C.) | 1.0 | |
| MinPressure (Bar) | 0.0 | |
| Max Pressure (Bar) | 100.0 | |

Waters Alliance 2690 Autosampler Initial Conditions

| Draw Speed | Fast |
|---|---|
| Needle Depth (mn) | 200.00 |
| End Wavelength (nm) | 254.00 |
| Sample Temperature | 1.2 |
| Pre-column Volume (μl) | 0.00 |

Waters Alliance 2690 Autosampler Initial Conditions

| Waters 996 PDA | |
|---|---|
| Start Wavelength (nm) | 200.00 |
| End Wavelength | 254.00 |
| Resolution (nm) | 1.2 |
| Sampling Rate (spectra/s) | 1.000 |
| Filter Response | 1 |
| Exposure Time (ms) | 500.00 |
| Acquisition Stop Time | Use pump stop time |

Waters 996 PDA Analog Channel 1

| Output Mode | Absorbance |
|---|---|
| Filter Type | Hamming |
| Wavelength (nm) | 220.00 |
| Bandwidth (nm) | 500.00 |
| Offset (AU) | 0.00 |
| Filter Response Time (s) | 5.00 |

Waters 996 PDA Analog Channel 2
Output Mode Off

The LC/MS spectrum of digoxin sample (attached) using photo diode array detector and UV 200 μm indicated presence of two compounds, having retention times 16.33 and 18.64 min and identical mass (779.81) required for digoxin. These peaks are designated as X (#1) and Y (#2) respectively.

Determination of Optical Rotation:

Optical rotation of digoxin samples peak #1 and peak #2 was determined using Perkin-Elmer Instrument. Peak #1 (1 mg/ml in alcohol) and peak #2 (1 mg/ml in alcohol) exhibited values of +17° and +3° respectively.

Determination of Molecular Weight by Mass Spectrometry:

Mass spectrometry was carried out for the above samples, peak #1 and peak #2. Both peaks indicated a molecular weight of 780, confirming the presence of digoxin in these samples.

Experimental Techniques for Determination of Differential Effects on Av Node Conduction and Cardiac Contractibility:

The guinea pig or rat is anesthetized with pentobarbital at 40 mg/kg IP, then connected to an EKG recorder. The animal is then placed on a small animal respirator. Aseptic surgical instruments are soaked in ethanol and then flamed. An incision is made in the chest wall to open the chest. A Walton Brody stain gauge arch is sutured to the right ventricle to measure contractile changes. The refractoriness of the AV node is determined by measuring the PR interval on the surface EKG. Graphs can be created from the data displaying the dose related change in the PR interval to the dose related contractile augmentation. Digoxin or chiral isolates are infused through the jugular vein at concentrations (from 0.1 μM and then to 10 μM) at a constant rate by infusion pump, to determine the differential effects on the AV node and cardiac contractibility. The infusions are continued to a 50% level augmentation in contractibility or the development of ventricular tachycardia. To maintain a consistent level of anesthesia, pentobarbital 20 mg/kg IP or 10 mg/kg IV for more immediate action at 30-minute intervals may be administered.

An assay of digoxin (and determination of its pharmacokinetics) from biological fluids, such as serum and urine, is routinely done following a variety of methods, such as fluorescence prolongation immunoassay (FPIA), microparticle enzyme immunoassay (MEIA), cheminluminescent assay (CLIA), radioimmunoassay, or anti-digoxin antiserum assay, for example.

Radioimmunoassay:

A radioimmunoassay was developed for each of the ionizable derivatives. In brief, antiserum from sheep, immunized and repeatedly boosted with digoxin-albumin conjugate, was used. [³H]digitoxin, 0.5 ng (New England Nuclear, sp act 10.9 Ci/mmol) and known concentrations of ASI-222 were incubated with an appropriate amount of antiserum for 20 minutes in 1.0 ml of a 1:1 mixture of phosphate-buffered saline (PBS) (0.15 M NaCl, 0.01 M Na₂HPO₄; adjusted to pH 7.4 with H₃PO₄) and normal plasma. Destran-coated charcoal was then added to remove free glycoside components, and the mixture was centrifuged for 20 min at 5,000 g. The supernatant phase was decanted into a scintillation spectrometer. Tritium internal standards were used for quench correction. The percent of antibody-bound [³H]digoxin or [³H]digitoxin, respectively, was plotted as a function of known concentrations of digoxin glucoronide or digitoxigenin sulfate to construct standard curves from which unknown values were determined. Modification of the ⁸⁶Rb method was used for serum analysis of digitoxin and cardioactive metabolites. The method has been found applicable to urine analysis without further modification. Urine pH did not influence the method. When testing the method for determination in bile, hemolysis of the erythrocytes occurred during incubation, probably because of bile acids. Different modifications were tried; 0.5 ml of 0.1 N sodium hydroxide was added to 1.0 ml of bile and subsequently extracted with 5 ml of dichloromethane. Four millimeters of the extract was then transferred to new glass tubes and evaporated. A second extraction was performed in the same manner, and the cichloromethane extract was transferred to new glass tubes containing dried extract from the first extraction. Addition of the ⁸⁶ Rb solution to the dried extracts and incubation with erythrocyte suspension were then performed in the usual manner.

Thin-Layer Plates (Thin Layer Chromatography):

A number of different types of plate were tried: silica gel according to Stahl (Merck, Darmstadt, G.F.R.) and Kieselguhr G (Merck) spread on glass plates; precoated silica gel F₂₅₄ TLC plates (Merck); pre-coated Kieselguhr F₂₅₄ TLC plates (Merck); fast-running silica gel F₂₅₄ TLC plates (Merck) (silica gel-Kieselguhr); and Chromagram 6064 cellulose sheets (Eastman-Kodak, Rochester, N.Y., USA).

Solvent Systems:

The following solvent systems were tested: chloroform-pyridine; cyclohexani-acetone-acetic acid; ethyl methyl ketone-chloroform-formamide; and 1,2-dicloroethane-methanol-water.

Impregnation:

Ascending-solvent impregnation with 10%, 15% or 20% formamide solution in acetone was used for many systems. The plates were impregnated overnight in sealed glass jars, and the test substances were applied directly after removal of the plates from the jars.

Standard Substances:

The following standard substances were used: digitoxin (DT-3), digitoxigenin bisdigitoxoside (DT-2), digitoxigenin monodigitoxoside (DT-1), digitoxigenin (DT-0), epi-digitoxigenin (Epi-DT-0), digoxin (DG-3), digoxigenin bisdigitoxoside (DT-2), digoxigenin monodigitoxoside (DT-0) and epi-digitoxigenin (Epi-DT-0), for example.

Detection Reagents:

Concentrated sulfuric acid in ethanol (1:4) was chosen for this purpose.

Extraction and Purification Procedure:

Five milliliters of serum of urine from patients undergoing treatment with digitoxin were shaken with 15 ml of dichloromethane (Merck) for 10 min; after separation of the phases by centrifugation, 10 ml of dichloromethane extract were evaporated to dryness at 50° on a water bath, then 3 ml of 70% ethanol was added to the residue and the dilution was washed twice with 0.7 ml portions of light petroleum (b.p. 40-60°) (Anal R; BDH, Poole, Great Britain). A portion (2.5 ml) of the ethanol extract was subsequently transferred to glass-stoppered conical tubes and evaporated in a water bath at 50°, in a stream of air. The dried extract was dissolved in 25 μl of this solution and applied to the thin-layer plates. The standards were applied in 5 μl of chloroform-methanol (50:50), and 15 μl of this solution were applied to the thin layer plates. The standards were applied in 5 μg amounts on both edges of each plate, with 6 or 7 samples in between. The R_{F} values of the standards were the same whether the compounds were added to serum, urine, or ethanol; a stock solution containing all the metabolites dissolved in pure ethanol was therefore used as standard.

Protein Binding Methodology:

Equilibrium dialysis proved to be the most satisfactory method for our purposes. Equilibrium was obtained after 16 hours when titrated glycoside was added to serum. When added to the buffer phase, equilibrium was reached after 60 hours. Distribution of digitoxin, for example, in buffer-buffer control, was around 50% (49.4 to 50.6%). Absorption of ³H-labeled glycoside to the dialysis membrane was 0.2% of the added ³H-glycoside. Digitoxin protein binding was dependent on albumin content. Addition of gamma globulin in concentrations of 0.9, 1.2, and 1.8 gm/dl did not affect protein binding. No trace of protein was found when adding concentrated perchloric acid to the buffer phase. Counting efficacy was the same whether serum or buffer aliquots were added to the counting fluid. The counting error for buffer samples containing the smallest amount of radioactivity was 1% to 1.5%. Chamber equilibrium dialysis gave similar results. Change of buffer did not alter the degree of protein binding. No significant change in digitoxin binding was found when the sera from 9 patients were dialyzed at 20° C. (mean, 96.7%) and at 37° C. (mean, 96.9%). Digoxin isolates will be used under similar conditions.

Free fraction of digitoxin increased with centrifugation time from 18% at 10 minutes to 37% at 80 minutes (500 g) without reaching plateau levels. At constant centrifugation time (60 minutes), free fraction increased from 5.9% at 135 g to 15.7% at 560 g.

Protein Binding of Digitoxin and Cardioactive Metabolites:

Triturated glycoside was available for the determination of DT-3, DG-3, and DG-1. The results obtained by adding unlabeled glycoside to serum, performing equilibrium dialysis and afterwards determining glycoside concentration in serum and buffer with the modified ⁸⁶Rb-method, were lower and less accurate with higher standard deviations than when using equilibrium dialysis alone.

In Vitro Drug Studies:

The ability to induce changes in digitoxin serum protein binding was tested for the following drugs: procainamide (5 mg/ml), phenytoin (50 mg/ml), heparin (5 IU/ml), and rifampicillin (4.5 μg/ml). None of these induced significant changes in the serum protein binding of digitoxin. Identical procedures will be used for digoxin isolates.

The method described by Ferren is used for analysis of digoxin centrations in serum by the FPIA procedure.

Reagents and Equipment:

All reagents, controls, and calibrators used with the TDX system were supplied or purchased from Abbott Laboratories. To minimize drug loss by binding of digoxin to glass and plastic, a serum based digoxin spiking solution was prepared. Digoxin (52 mg; Aldrich Chemical) was dissolved in 50 mL tetrahydofuran:water (water:20 v/v) and mixed for 2 hours. One milliliter of the solution was transferred to 1 L of Abbott TDX buffer (0.1 M phosphase and 0>1% sodium azide, containing a protein stabilizer) and was mixed for two days at room temperature. This stock digoxin in buffer (0.5 mL) was added to 10 mL of normal human serum (zero calibrator obtained from Abbott). The final digoxin concentration of the serum based spiking solution was 49.5 μg/m: this solution was used to prepare samples for precision and accuracy studies.

Precision:

Assay precision was determined using blood bank plasma that was dialyzed (against Renalyte Hemodialysis Fluid, Cobe Laboratories), allowed to clot, and spiked with digoxin to yield concentrations of 0.35, 0.64, 1.32, 2.08, and 3.75 ng/ml. Nine or ten replicates of each concentration were assayed in a single run. Between runs, precision was calculated from Abbott controls which had concentrations of 0.75, 1.50, and 3.48 ng/mL. The between-run precision data were collected over a one-month period under routine operating conditions, including the daily rotation of more than ten technologists using the TDX. The protocol followed was as described in the TDX protocol manual.

Recovery and Sensitivity:

To 1 mL of zero calibrator was added 20 μL of digoxin serum based spiking solution, providing samples with digoxin concentrations of 0.97 ng/mL and 4.50 ng/mL, respectively. The percent recovery was calculated from the ratio (X100) of the TDX measured concentration to the known digoxin concentration. Sensitivity was evaluated by comparing Abbott serum based zero calibrator to patient serum spiked to a concentration of 0.2 ng/mL.

Interference and Cross-Reactivity:

Sera with elevated bilirubin (27 mg/dL), hemoglobin (0.7 g/dL), and triglycerides (836 mg/dL) were spiked with digoxin, and the percent recovery was determined. In cross-reactivity studies, sera containing digitoxin (50 mg/mL and 25 mg/mL) and spironolactone (20 μg/mL and 10 μg/mL) were assayed on the TDX.

Correlation Study:

Samples were analyzed using an antibody-coated tube RIA (Clinical Assays). All standards, controls, and patient specimens were assayed according to the manufacturer's protocol. The samples were then assayed on the TDX, and least squares regression was used to determine the correlation coefficient ® slope, y-intercept, and the standard deviation of the regression line (_{y/x}). Deming's Regression Method was also used to evaluate the influence of greater impression in the RIA reference method compared with the TDX.

Digoxin Studies: (Biology)

The effect of digoxin and one of the two chiral isomers was evaluated in the guinea pig. Guinea pigs were anesthetized with pentobarbital. A vein was canulated for IV drug administration. The animal was intubated and its chest opened. The heart was suspended in a pericardial cradle and a Walton Brody strain-gauge arch was sutured to the left ventricle to measure contractibility. An EKG was continuously recorded. Conduction through the AV node was determined by measuring the PR interval on the surface EKG. Digoxin or Isomer X was infused at 6 μg/kg/min continuously. In this model, digoxin caused a progressive increase in PR interval as well as an increase in cardiac contractibility as measured by the strain-gauge arch. The isomer caused a progressive increase in the PR interval but essentially no change in cardiac contractibility.

Conclusion:

One of the two isolates (chiral) of digoxin behaves differently in terms of contractibility action and has disperate pharmacodynamic action from digoxin in terms of its augmentation of cardiac contractibility. These results (regarding percentage changes in contractibility and PR interval) are summarized in the following figures.

What is claimed is:

1. Optically active isomers of digoxin (II) having the formula:

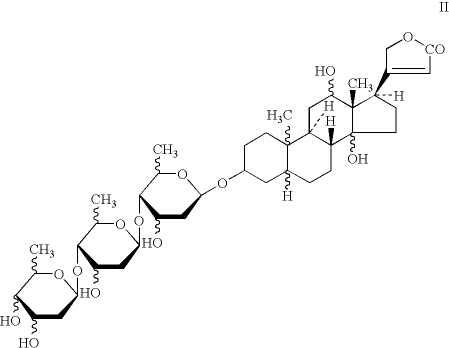

2. Optically active isomers of digoxin that differ in optical activity and that possess differences in degrees of biologic effect on the AV node conduction, heart rate slowing and cardiac contractile augmentation.

3. The method of treatment of supar ventricular arrhythmia which comprises administering to a patient in need of such treatment a chiral isolate or mixture of isolates of digoxin that predominantly effects conduction or refractoriness at the AV node with less or no effort on cardiac contractibility.

4. The method of treatment of congestive heart failure which comprises administering to a patient in need of such treatment a chiral isolate or mixture of isolates of digoxin that augment cardiac contractibility but has a less or no effect on heart rate slowing or PR conduction prolongation (AV Node refractions and conduction).

5. An isolate or mixture of isolates of digoxin that has less or no contractibility augmenting action but still possesses the ability to slow conduction at the AV node such that the composition of isomers may be effective in slowing the ventricular response in atrial fibrillation.

6. The optically active isomers of digoxin or mixtures of optically active isomers of digoxin still possessing cardiotonic activity, while no longer causing or causing a reduced incidence of the adverse effects known to occur with the clinical uses of chronic digoxin therapy.
